(19) United States
(12) Patent Application Publication
(10) Pub. No.: US 2009/0209842 A1
(43) Pub. Date: Aug. 20, 2009

(54) MRI GRADIENT COIL ASSEMBLY WITH REDUCED ACOUSTIC NOISE

(75) Inventors: Adrianus Hendrikus Koevoets, Eindhoven (NL); Georgo Zorz Angelis, Eindhoven (NL); Nicolaas Bernardus Roozen, Eindhoven (NL); Gerardus Nerius Peeren, Eindhoven (NL); Cornelis Leonardus Gerardus Ham, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/307,623

(22) PCT Filed: Jul. 3, 2007
(86) PCT No.: PCT/IB2007/052583

§ 371 Date: Jan. 6, 2009

(30) Foreign Application Priority Data
Jul. 7, 2006 (EP) 06116760.7

(51) Int. Cl. A61B 5/055

(52) U.S. Cl. 600/410

(57) ABSTRACT

The invention relates to a magnetic resonance imaging system which comprises means for generating a static magnetic field and a gradient coils system for generating a time varying magnetic gradient field by use of a first electrical current and a second electrical current. The gradient coils system is located in the magnetic field and the gradient coils system has a plurality of vibrational modes. Lorentz forces are generated due to the interaction of the first and/or second electrical currents with the superposition of the static magnetic field and the magnetic gradient field. The gradient coils system and/or the first electrical current are adapted so that the integral of the in-products of said Lorentz forces and a vibrational mode of said plurality of vibrational modes is at a value close to zero, wherein said in-products are determined for all points of the gradient coils system, and wherein the integral is determined by summing the in-products determined for all the points. As the above mentioned integral is close to zero or preferably zero, the Lorentz forces are not able to excite the vibrational mode (for example the lowest order bending mode) of the gradient coils system. Thus acoustical noise that is generated by a vibrating gradient coils system is reduced and the comfort for a patient that is examined by the magnetic resonance imaging system is therefore enhanced.

### FIELD OF THE INVENTION

The present invention relates to a magnetic resonance imaging system and to a gradient coils system for the magnetic resonance imaging system in general and to a magnetic resonance imaging system comprising a gradient coils system that is adapted and operated so that the acoustic noise generated by the gradient coils system is minimized. In other aspects, the invention relates to a method of reducing acoustic noise generated by the magnetic resonance imaging system and to a computer program product that is adapted to perform steps of the method in accordance with the invention.

### BACKGROUND AND RELATED ART

Magnetic gradient coils are a pre-requisite for nuclear magnetic resonance imaging (P. Mansfield P. and P. G. Morris, NMR Imaging in Biomedicine, Academic Press, New York, 1982) and also for use in a range of nuclear magnetic resonance applications including diffusion studies and flow. In nuclear magnetic resonance imaging the acoustic noise associated with rapid gradient switching combined with higher static magnetic field strengths is at best an irritant and at worst could be damaging to the patient. Some degree of protection can be given to adults and children by using ear defenders. However, for fertile scanning and in veterinary applications, acoustic protection is difficult if not impossible.

Several attempts have been made to ameliorate the acoustic noise problem. For example, by lightly mounting coils on rubber cushions, by increasing the mass of the total gradient assembly and by absorptive techniques in which acoustic absorbing foam is used to deaden the sound. Acoustic noise cancellation techniques have also been proposed which rely on injection of anti-phase noise in headphones to produce a localized null zone. These methods are frequency and position dependent and could possibly lead to accidents where, rather than cancel the noise, the noise amplitude is doubled.

The document U.S. Pat. No. 5,990,680 proposes a method for active control of the acoustic noise generated by a magnetic gradient coil design. An active acoustically controlled magnetized-coil system is described in the above cited document which is adapted to be placed in a static magnetic field. The coil comprises a plurality of first electrical conductors and a plurality of at least second electrical conductors. The first and at least the second conductors are mechanically coupled by means of at least one block of material with a predetermined acoustic transmission characteristic and the first and at least the second conductors are spaced at a predetermined distance apart. The coil further comprises first electrical current supply means for supplying a first alternating current to the first electrical conductors, at least a second electrical current supply means for supplying at least a second alternating current to the at least second electrical conductor. The first and at least second currents are characterized in that they have different and variable amplitudes and different and variable relative phases, both these features being determined by the acoustic characteristics of the material and by its geometry and the predetermined distance.

A disadvantage of the method and system proposed in U.S. Pat. No. 5,990,680 is that the first electrical current supply means and the at least second electrical supply means are fairly complex electronic systems as they have to provide the first and at least second currents, whereby both have to be adapted in amplitude and relative phase with respect to each other.

There is therefore a need for an improved magnetic resonance imaging system, for an improved gradient coils system, and for a method of reducing the acoustic noise generated by the magnetic resonance imaging system.

### SUMMARY OF THE INVENTION

In one aspect, the invention relates to a magnetic resonance imaging system (MRI system). The magnetic resonance imaging system comprises means for generating a static magnetic field and a gradient coils system for generating a time varying magnetic gradient field by use of a first electrical current and a second electrical current, wherein the gradient coils system is located in the static magnetic field and wherein the gradient coils system has a plurality of vibrational modes. Lorentz forces are generated along the gradient coils system due to the interaction of the first and/or second electrical currents with the superposition of the static magnetic field and the magnetic gradient field, wherein the gradient coils system and/or the first electrical current are adapted so that the integral of the in-products of said Lorentz forces and a vibrational mode of said plurality of vibrational modes is at a value close to zero, wherein said in-products are determined for all points of the gradient coils system, and wherein the integral is determined by summing the in-products determined for all points.

The gradient coils system which is also known by the terms gradient magnet system and gradient system is able to perform a mechanical motion in the magnetic resonance system. The mechanical motion corresponds to a vibration or oscillation of the gradient coils system which is describable by a superposition of the vibrational modes of the gradient coils system.

The gradient coils system is from a continuous dynamics point of view a continuous system and the gradient coils system therefore comprises a plurality of vibrational modes, whereby each mode is characterized by a specific mode shape and a mode frequency. The mode frequency is also referred to in the following as oscillation frequency. The vibrational modes depend for example on the design of the gradient coils system, on the material of the coil system, and on the way the gradient coils system is mounted to the magnetic resonance imaging system.

The vibrations of the gradient coils system are a major source of the acoustic noise generated when the MRI system is operated. The vibrations of the gradient coils system are forwarded by different spreading paths to the surface of the MRI system. The surface velocity determines the transmission of the mechanical vibration or oscillation into the acoustic oscillation and is composed of the superposition of the oscillations of the individual transmission paths. The surface velocity determines the noise generated by the device in connection with the geometry of the surface. If the excitation of the vibrational modes can be prevented as proposed here, then the acoustic output of the MRI system can be reduced, maybe even dramatically.

The vibrational modes can be excited by Lorentz forces that are generated due to the interaction of the first and/or second currents flowing through the coil system and the superposition of the static magnetic field and the magnetic gradient field at the corresponding locations of the current flow. The magnetic resonance imaging system is particularly advantageous as the gradient coils system and/or the first electrical current are adapted so that the integral of the in-products of the Lorentz forces and the vibrational mode is at a value close to zero or preferably even zero, wherein the in-products are determined for all points of the gradient coils system, and wherein the integral is determined by summing the in-products determined for all points.

The in-product is a mathematical operation that can be performed on two vectorial quantities and that yields a scalar value. The in-product is also referred to as scalar product or inner product. The above mentioned integral of the in-products of the Lorentz forces and the vibrational mode can be determined in the following way: The Lorentz forces and a vibrational mode can be described as vectorial quantities on each point of the gradient coils system. The in-product of the Lorentz force and the vibrational mode can therefore be determined on each point of the gradient coils system where the Lorentz forces and the vibrational mode have non-zero values. These in-products can be summed (integrated) over all points of the gradient coils system which yields in accordance with the invention an integral value for the in-products of the Lorentz force and the vibrational mode which is close to zero or even zero. The Lorentz forces are therefore balanced along the gradient coils system and are merely or not at all able to excite a vibrational mode. Hence vibrations of the gradient coils systems are not induced or they are induced at a reduced level. As a result, the acoustic output or noise of the gradient coils system which is generated due to the vibrations of the gradient coils system is reduced and thus the comfort of patients examined by such a magnetic resonance imaging system is increased. Preferably, the above mentioned vibrational mode relates to the lowest order bending mode of the gradient coils system as this vibrational mode is the dominant vibrational mode and thus the major source of noise.

In accordance with an embodiment of the invention, the gradient coils system comprises an inner coil and an outer coil, wherein the inner and outer coil are mechanically coupled, wherein the outer coil is operated by use of the first electrical current, wherein the inner coil is operated by use of the second electrical current, wherein the first electrical current is adapted so that the integral of the in-products of said Lorentz forces and a vibrational mode of said plurality of vibrational modes is at a value close to zero, wherein the in-products are determined for all points of the gradient coils system, and wherein the integral is determined by summing the in-products determined for all points. In the embodiment described here, the first electrical current is applied to the outer coil, and the second electrical current flows through the inner coil.

Typical gradient coils systems comprise an inner and an outer coil. The inner coil is also called primary coil and the outer coil is sometimes called secondary coil. The inner coil is situated closer to the examination volume of the MRI system and the outer coil is placed between the inner coil and the main magnet. The inner and outer coils are usually mechanically connected as both reside for example in an epoxy resin. The main magnet is used to generate the static magnetic field in the examination volume (the examination space of the MRI system). The main magnet is usually a superconducting magnet that resides in a cryostat as it must be cooled to cryogenic temperatures.

The inner gradient coil is further employed for the generation of the desired magnetic gradient field at the examination volume. The magnetic field generated by the inner coil can induce eddy currents in the cryostat which lead to an undesired heating of the cryostat or in other conducting surfaces of the MRI system. The eddy currents might also generate magnetic stray fields that might cause disturbances in the examination volume. The outer coil is used to generate a magnetic gradient field at the cryostat which compensates the magnetic field of the inner coil so that the induction of eddy currents is prevented or at least reduced.

When operating a gradient coils system, it is aimed for a) the generation of the desired time-varying magnetic gradient field in the examination volume and b) the prevention of the generation of eddy currents. As mentioned above, the outer coil is used to generate a magnetic gradient field so that the induction of eddy currents is prevented. For this, the first electrical current flowing through the outer coil is adapted accordingly so that condition b) is fullfilled.

Furthermore, the second electrical current flowing through the inner coil is adapted in order to fulfill the above mentioned condition a).

It is further aimed for c) a reduction of the noise generated by the gradient coils system. According to the embodiment described above, condition c) is achieved by adapting the first current so that the acoustic noise produced by the gradient coils system is minimized. This usually leads to an increased generation of eddy currents, and hence (condition b)) will not be optimally fulfilled any more. This can also lead to a magnetic gradient field in the examination volume that is not optimal. Condition a) might therefore also not be optimally fulfilled.

Measurements have however shown that the heating of the cryostat due to the induced eddy currents is still acceptable when the first electrical current is adapted in order to minimize the acoustic noise. Further, measurements have shown that most scans (a scan refers to the processes of examining a patient by use of a sequence of varying gradient magnetic fields) that involve specific gradient magnetic fields in the examination space can be carried out without loss of resolution when condition a) is not optimally fulfilled.

In accordance with an embodiment of the invention, the magnetic resonance imaging system further comprises a first amplifier and a second amplifier. The first amplifier is electrically connected with the outer coil and is able to provide the first electrical current. The second amplifier is electrically connected to the inner coil and is able to provide the second electrical current. Thus, the first electrical current running through the outer coil and the second electrical current flowing through the inner coil are provided by the first and second amplifiers.

In accordance with an embodiment of the invention, the outer coil is adapted to comprise a plurality of windings, and the first amplifier is a low power amplifier. Usually, a high power amplifier is employed for driving the first current through the outer coil. However, if the outer coil is adapted so that it comprises a plurality of windings, the magnetic field generated by the inner coil induces a voltage in the outer coil as the magnetic field is time varying (Faraday's induction law). Due to the induced voltage, an induced current is generated that flows through the outer coil. Then, only a low power amplifier is required in order to be able to adjust the first current which corresponds to the sum of the induced current and the current provided by the low power amplifier.

In accordance with an embodiment of the invention, the gradient coils system comprises an inner coil, an outer coil, and a force compensation coil, wherein the inner, outer and force compensation coil are mechanically connected, wherein the force compensation coil is operated by use of the first electrical current, and wherein the inner and outer coils are operated by use of the second electrical current. According to this embodiment of the invention, an extra coil, the so called force compensation coil, is attached to the gradient coils system. The first electrical current is flowing through the force compensation coil and the first current is adapted so that the integral of the in-products of said Lorentz forces and the dominant vibrational mode of said plurality of vibrational modes is at a value close to zero, wherein the in-products are determined for all points of the gradient coils system, and wherein the integral is determined by summing the in-products determined for all points.

In accordance with an embodiment of the invention, the force compensation coil is placed in the vicinity of the outer coil on the opposite side of the inner coil. The force compensation coil is therefore placed at the outer side of the outer coil on the opposite side of the inner coil. Alternatively the force compensation coil is placed to the inside of or underneath the outer coil or on the top of the outer coil.

In accordance with an embodiment of the invention, the magnetic resonance imaging system further comprises a first amplifier and a second amplifier, wherein the inner and the outer coils are electrically connected with the second amplifier, wherein the force compensation coil is electrically connected with the first amplifier, wherein the first amplifier provides the first electrical current and wherein the second amplifier provides the second electrical current. The first electrical current through the force compensation coil is relative small with respect to the second electrical current. Hence, the first electrical current can be provided by a low power amplifier which is relative inexpensive with respect to the high power amplifier used for the provision of the second current.

In accordance with an embodiment of the invention, the vibrational mode corresponds to the first order bending mode of the gradient coils system. The first order bending mode is also referred to as banana mode as the shape of the gradient coils system resembles to the form of a banana at the points of maximal elongation. The lowest order bending mode is the major source of noise generated from the vibration of the gradient coils system. Hence, if the integral over the gradient coils system of the in-products of the Lorentz forces and the lowest order bending mode is at a value close to zero, then the lowest order bending mode is merely or even not at all excited and thus, the noise is reduced, maybe even dramatically. The integral over the gradient coils system of the in-products of the Lorentz forces and another vibrational mode which is not the lowest order bending mode can additionally become zero. As mentioned above, vibrations in the lowest order bending mode are the major source of noise. Thus by not exciting the lowest order bending mode, the largest net effect in reducing the noise will be achieved. By additionally preventing the excitation of the other vibrational mode, the acoustic noise might therefore be further reduced, but less dramatically.

In accordance with an embodiment of the invention, the inner coil comprises an inner x-coil and the outer coil comprises an outer x-coil, wherein the first current is flowing through the outer x-coil and wherein the second current is flowing through the inner x-coil, wherein the first amplifier is electrically connected with outer x-coil, and wherein the second amplifier is electrically connected with the inner x-coil.

In accordance with an embodiment of the invention, the inner coil comprises an inner y-coil and the outer coil comprises an outer y-coil, wherein the first current is flowing through the outer y-coil and wherein the second current is flowing through the inner y-coil, wherein the first amplifier is electrically connected with the outer y-coil, and wherein the second amplifier is electrically connected with the inner y-coil.

In accordance with an embodiment of the invention, the inner coil comprises an inner z-coil and the outer coil comprises an outer z-coil, wherein the first current is flowing through the outer z-coil and wherein the second current is flowing through the inner z-coil, wherein the first amplifier is electrically connected with the outer z-coil, and wherein the second amplifier is electrically connected with the inner z-coil.

Each, the inner coil as well as the outer coil comprise therefore a x-coil, a y-coil, and a z-coil which are used to generated a magnetic gradient field that is directed into the x-, y-, or z-direction, respectively. The first and second currents are provided to all three, the x-coils, the y-coils and the z-coils, whereby the first currents are adapted so that the integral of the in-products of said Lorentz forces and of the vibrational mode is at a value close to zero or even zero, wherein the in-products are determined for all points of the gradient coils system, and wherein the integral is determined by summing the in-products determined for all points.

The amplitudes and frequencies of the first currents and the second currents applied to the x-, y-, and z-coils might, of course, be different from coil to coil.

In accordance with an embodiment of the invention, the inner coil comprises an inner x-coil and the outer coil comprises an outer x-coil, wherein the gradient coils system further comprises a force compensation coil, wherein the second current is flowing through the inner and outer x-coils, and wherein the first current is flowing through the force compensation coil.

In accordance with an embodiment of the invention, the inner coil comprises an inner y-coil and the outer coil comprises an outer y-coil, wherein the gradient coils system further comprises a force compensation coil, wherein the second current is flowing through the inner and outer y-coils, and wherein the first current is flowing through the force compensation coil.

In accordance with an embodiment of the invention, the gradient coils system comprises a z-coil and a force compensation coil, wherein the z-coil and the force compensation coil are mechanically coupled, wherein the force compensation coil is operated by use of the first electrical current, and wherein the z-coil is operated by use of the second electrical current.

In accordance with an embodiment of the invention, the magnetic resonance imaging system further comprises a first amplifier and a second amplifier, wherein the z-coil is electrically connected with the second amplifier, wherein the force compensation coil is electrically connected with the first amplifier, wherein the first amplifier provides the first electrical current, and wherein the second amplifier provides the second electrical current.

In accordance with an embodiment of the invention, the vibrational mode corresponds to the breathing mode of the z-coil.

In accordance with an embodiment of the invention, the first and the second electrical currents are time varying electrical currents.

In accordance with an embodiment of the invention, the spatial distribution of the conductors of the gradient coils system is adapted. In the preceding embodiments, the design of the gradient coils system has not been changed to a great extent (only more windings in the outer coil or a force compensation coil have been added). The first electrical current has been adapted so that the integral of the in-products of said Lorentz forces and a vibrational mode of said plurality of vibrational modes is at a value close to zero, wherein the in-products are determined for all points of the gradient coils system, and wherein the integral is determined by summing the in-products determined for all points. As a result, the vibrations of the gradient coils system could be reduced. In the embodiment described here, the spatial distribution of the conductors of the gradient coils system is adapted. This has the advantage, that the gradient coils system can be optimized in order to fulfill the conditions of a) the generation of the desired magnetic gradient field in the examination volume of the MRI system, b) no or nearly-no generation of eddy currents and c) minimizing the noise generated by the gradient coils system.

In accordance with an embodiment of the invention, the magnetic resonance imaging system further comprises a control system, wherein the control system is able to adapt the first time varying electrical current so that the integral of the in-products of said Lorentz forces and a vibrational mode of said plurality of vibrational modes is at a value close to zero, wherein said in-products are determined for all points of the gradient coils system, and wherein the integral is determined by summing over all said points.

In a second aspect, the invention relates to a gradient coils system for a magnetic resonance imaging system, wherein the magnetic resonance imaging system comprises means for generating a static magnetic field, and wherein the magnetic resonance imaging system is adapted to receive the gradient coils system, wherein the received gradient coils system is located in the static magnetic field, wherein the gradient coils system has a plurality of vibrational modes, wherein the gradient coils system is adapted to generate a magnetic gradient field, wherein the magnetic gradient field is activated by a first electrical current and a second electrical current, wherein Lorentz forces are generated along the gradient coils system due to the interaction of the first and second electrical currents with the superposition of the static magnetic field and the magnetic gradient field, wherein the gradient coils system and/or the first electrical current are adapted so that the integral of the in-products of said Lorentz forces and a vibrational mode of said plurality of vibrational modes is at a value close to zero, wherein the in-products are determined for all points of the gradient coils system, and wherein the integral is determined by summing the in-products determined for all points.

In accordance with an embodiment of the invention, the gradient coils system comprises an inner coil and an outer coil, wherein the inner and outer coil are mechanically coupled, wherein the outer coil is operated by use of the first electrical current, wherein the inner coil is operated by use of the second electrical current, wherein the first electrical current is adapted so that the integral of the in-products of said Lorentz forces and a vibrational mode of said plurality of vibrational modes is at a value close to zero, wherein said in-products are determined for all points of the gradient coils system, and wherein the integral is determined by summing over all said points.

In accordance with an embodiment of the invention, the gradient coils system comprises an inner coil, an outer coil, and a force compensation coil, wherein the inner, outer and force compensation coils are mechanically connected, wherein the force compensation coil is operated by use of the first electrical current, wherein the inner and outer coils are operated by use of the second electrical current.

In a third aspect, the invention relates to a method of reducing acoustic noise generated by a magnetic resonance imaging system, wherein the magnetic resonance imaging system comprises means for generating a static magnetic field in a examination volume of the magnetic resonance imaging system, and a gradient coils system for generating a time varying magnetic gradient field in the examination volume, wherein the time varying magnetic gradient field is activated by a first electrical current and a second electrical current flowing through the gradient coils system, wherein the method comprises the step of setting the first electrical current to a time-varying first amplitude and the second electrical current to a time-varying second amplitude, wherein the first and second amplitudes are set in order to generate desired time varying magnetic gradient fields in the examination volume. The method in accordance with the invention further comprises the step of changing the first amplitude of the second electrical current to a time-varying third amplitude so that a mechanical motion of the gradient coils system is minimized, wherein the mechanical motion is induced by the interaction of the first and second electrical currents with the superposition of the magnetic gradient field and the static magnetic field.

An implementation of the method of the invention further involves determining said mechanical motion of said gradient coils system.

An implementation of the method of the invention is applied to the gradient coils system which comprises an inner and an outer coil, wherein said inner coil and outer coil are mechanically coupled, wherein said first electrical current is applied to said outer coil and wherein said second electrical current is applied to said inner coil.

An implementation of the method of the invention is applied to the gradient coils system which comprises an inner coil, an outer coil (718), and a force compensation coil (716), wherein said inner coil, said outer coil and said force compensation coil are mechanically connected, wherein said first electrical current (722) is applied to said force compensation coil, and wherein said second electrical current (728) is applied to said inner and outer coil.

An implementation of the method of the invention is applied to the gradient coils system which has a plurality of vibrational modes, wherein the mechanical motion corresponds to a vibration which can be described by a superposition of the vibrational modes of said plurality of vibrational modes.

In particular, mechanical motion is measured by a sensor (714).

For example, the sensor (714) is an accelerometer or a vibration sensor, said sensor being fixed to said gradient coils system.

For example the mechanical motion is determined by measuring an acoustic power generated by the gradient coils system.

In practice, the acoustic power is measured by a microphone.

For example, a temperature sensor is mounted to the gradient coils system, and wherein said first time-varying first amplitude is changed to said third amplitude in response to a change of temperature of the gradient coils system, wherein said change of temperature is measured by said temperature sensor.

In a fourth aspect, the invention relates to a computer program product for reducing acoustic noise generated by a magnetic resonance imaging system, wherein the magnetic resonance imaging system comprises a examination volume, means for generating a static magnetic field in the examination volume, and a gradient coils system for generating a time varying magnetic gradient field in the examination volume, wherein the time varying magnetic gradient field is activated by a first electrical current and a second electrical current flowing through the gradient coils system, wherein the computer program product comprises computer executable instructions, and wherein the instructions are adapted to perform the steps of setting the first electrical current to a first amplitude and the second electrical current to a second amplitude, and of changing the first amplitude of the first electrical current to a third amplitude so that a mechanical motion of the gradient coils system is minimized, wherein the mechanical motion is induced by the interaction of the first and second electrical current with the superposition of the magnetic gradient field and the static magnetic field.

An implementation of the method of the invention involves changing the first electrical current back from the third amplitude to the first amplitude for the case when the magnetic resonance imaging system is to be operated with the desired time-varying magnetic gradient field.

A practical implementation of the computer program of the invention further comprising instructions for determining a mechanical motion of said gradient coils system.

The aspects of the invention described above will become even more apparent from and elucidated with reference to the embodiments described hereinafter.

### BRIEF DESCRIPTION OF THE DRAWINGS

In the following preferred embodiments of the invention will be described in greater detail by way of example only making reference to the drawings in which:

FIG. 1 shows schematically a sectional view of a magnetic resonance imaging system,

FIG. 2 shows a sectional view of an embodiment of a gradient coils system,

FIG. 3 illustrates the vibrational motion of the gradient coils system in the lowest order vibrational mode,

FIG. 4 depicts schematically an embodiment of a gradient coils system,

FIG. 5 shows schematically another embodiment of a gradient coils system,

FIG. 6 shows a flow diagram that illustrates the basic steps performed by the method in accordance with the invention,

FIG. 7 shows a block diagram of a magnetic resonance imaging system, and

FIG. 8 shows a cross section through a magnetic resonance imaging system.

### DETAILED DESCRIPTION

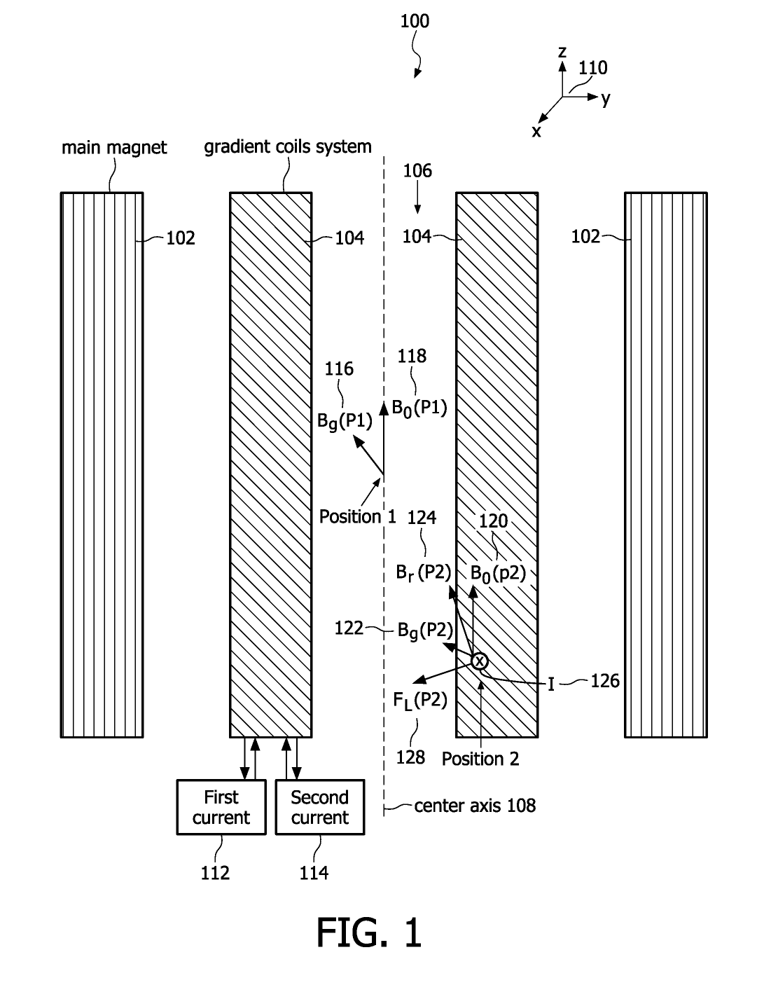

FIG. 1 shows schematically a sectional view of a magnetic resonance imaging system 100. A coordinate system 110 is defined in FIG. 1. According to the coordinate system 110, the sectional view refers to a cut through the magnetic resonance imaging system 100 in the yz-plane. The magnetic resonance imaging system 100 comprises an examination volume 106, a gradient coils system 104, and a main magnet 102. The examination volume 106 serves as an examination space, e.g., for a patient. The examination volume 106 comprises a center axis 108. The main magnet 102 and the gradient coils system 104 are cylindrically symmetric with respect to the center axis 108. The main magnet 102 and the gradient coils system 104 are depicted for simplicity reasons as rectangulars. The actual shapes of the main magnet 102 and the gradient coils system 104 are however much more complex.

The main magnet 102 is for example a super conducting magnet that is used to generate a static magnetic field B₀ in the examination volume 106 (vectorial quantities are denoted by bold letters). As an example, the magnetic field B₀ (P1) 118 is depicted at the point P1 which is a point on the center axis 108.

The gradient coils system 104 is used to generate a time varying magnetic gradient field B_{g} in the examination volume 106. As an example, the magnetic gradient field B_{g} (P1) 116 is plotted in FIG. 1 at the position P1.

The gradient coils system 104 has a plurality of vibrational modes. A vibration of the gradient coils system 104 can therefore be described as a superposition of the vibrational modes of the gradient coils system.

The gradient coils system 104 is placed at the inside of the main magnet 102. The magnetic field B₀ produced by the main magnet 102 is therefore also present at the location of the gradient coils system 104 (though the magnitude might be smaller there than in the examination volume 106). The time varying magnetic gradient field B_{g} is generated by the gradient coils system when a first and second electrical current 112 and 114 is flowing through the gradient coils system 104. Lorentz forces are generated along the gradient coils system due to the interaction of the first and/or second electrical currents 112 and 114 and the superposition of the static magnetic field and the magnetic gradient field.

As an example, the magnetic field B₀ (P2) 120 and the magnetic gradient field B_{g} (P2) 122 are shown in FIG. 1 at the position 2. The vectorial sum of the magnetic fields 120 and 122 yields the resulting magnetic field Bᵣ (P2) 124 at position 2. Usually, the magnitude of the main magnetic field B₀ is much larger than the magnitude of the magnetic gradient field B_{g} and the effect of this field can be neglected. The resulting magnetic field Bᵣ will therefore be approximately equal to B₀. The current I 126 which might correspond to the first current 112, to the second current 114 or to the (directed) sum of the first current 112 and second current 114 flows through the gradient coils system 104 along a path with length Δl (Δl is directed along the x-axis) at the position 2.

The Lorentz force F_{L}, 128 that is acting on the gradient coils system along the path with length Δl is then given by:

F_{L}=I·Δl×Bᵣ,

wherein the cross denotes the cross product between the vector Bᵣ(P2) (here a vector in the yz-plane) and Δl (here directed along the x-axis). The Lorentz force F_{L} (P2) 128 at the position 2 is therefore given by the cross product of the current I 126 passing through the path element Δl and the resulting magnetic field Bᵣ (P2) 124.

In general, the Lorentz forces acting on the gradient coils system can be denoted by F_{L}(x,y,z,t) as their magnitude and direction depend on the coordinates x, y, and z and on the time t. The Lorentz Force F_{L}(x,y,z,t) can then be determined as described above for position 1 for all points x, y, z of the gradient coils system (if no Lorentz force is present at a point, then F_{L}=0 at this point.

Furthermore, a vibrational mode n of the plurality of vibrational modes of the gradient coils system can be described by a vectorial quantity Vₙ(x,y,z) whose magnitude and direction are dependent on x, y, and z.

The in-product (scalar product) between the Lorentz force and the vibrational mode n is given by <Vₙ(x,y,z), F_{L}(x,y,z,t)> for each position along the gradient coils system. The integral can then be determined by

$${\int }_{g . c . s .} < V ( x , y , z ) {F}_{L} ( x , y , z , t ) > ,$$

wherein the integral is taken over all points of the gradient coils system (g.c.s.).

The Lorentz force depends on the resulting magnetic field Bᵣ and on the current I which corresponds to the first or to the second current (depending where on the gradient coil system the current I is considered). The integral as given above depends therefore on the first current. The first current 112 that is flowing through the gradient coils system 104 can then be adapted by changing its amplitude so that the above given integral of the in-products of Lorentz forces and the vibrational mode is minimized toward zero or becomes even zero. If the integral is at a value which is close to or ideally at zero, the Lorentz forces generated due to the interaction of the first and/or second current and the magnetic fields are nearly or even completely balanced along the gradient coils system. They are therefore unable or nearly unable to excite a vibrational mode of the plurality of modes for which the above mentioned integral is zero. As a result, the vibrations of the gradient coils system are reduced or even suppressed. Hence the acoustic noise that is generated by the vibrating gradient coils system 104 is reduced or even suppressed.

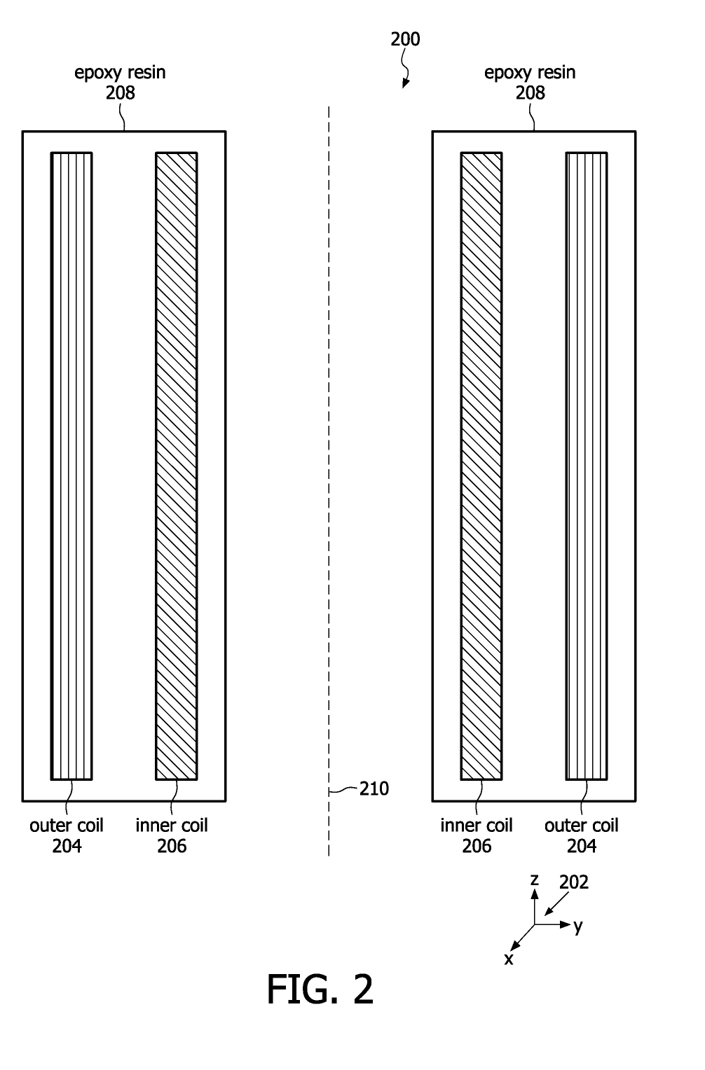

FIG. 2 shows schematically a sectional view of a gradient coils system 200. A coordinate system 202 is defined in FIG. 2. According to the coordinate system 202 the gradient coils system is depicted via a cut through the yz-plane. The gradient coils are placed around a center axis 210 of the MRI system which corresponds to the center axis of the examination volume. The gradient coils system comprises an outer coil 204 and an inner coil 206. The inner coil 206 and the outer coil 204 are placed in an epoxy resin 208 so that the inner coil and the outer coil are mechanically connected. The epoxy resin 208, the inner coil 206, and the outer coil 204 are depicted by rectangulars. This is an oversimplification. The actual shapes are, of course, much more complex.

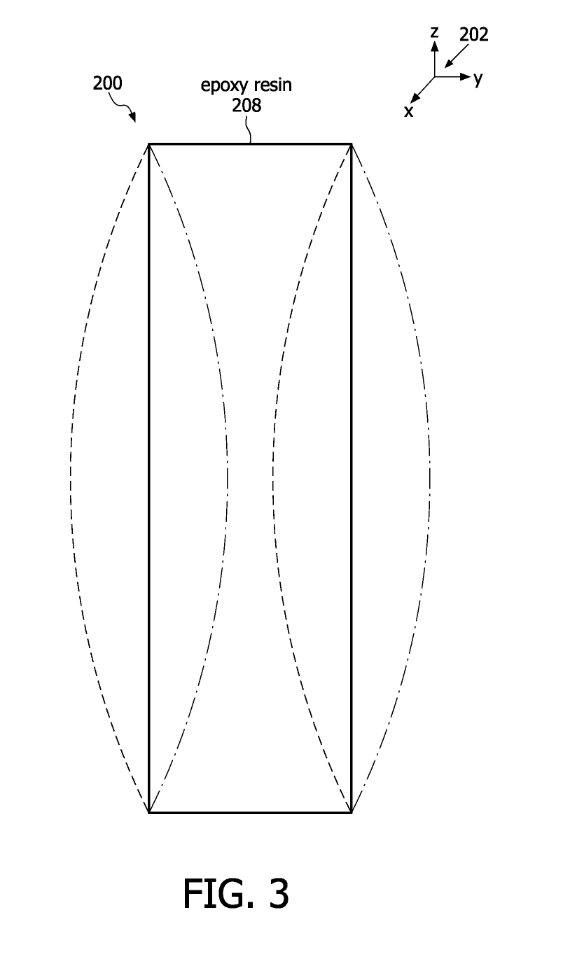

FIG. 3 illustrates the vibrational motion of the gradient coils system 200 for the case when the gradient coils system is vibrating in the lowest order vibrational mode. FIG. 3 shows the epoxy resin 208 as introduced in FIG. 2. The inner and outer coils shown in FIG. 2 are for simplicity reasons not depicted anymore in FIG. 3. The solid lines in FIG. 3 reflect the shape of the epoxy resin 208 for the case when the gradient coils system 200 is at rest or reaches its minimal elongation. The dashed lines reflect the shape of the epoxy resin when the motion of the gradient coil 200 reaches its maximum elongation to the left and the dashed dotted lines reflect the shape of the epoxy resin 208, when the maximum elongation to the right is reached. As can be seen, the lowest order mode corresponds therefore to a mode with a node on each end of the epoxy resin 208 and with an anti-node at the center of the epoxy resin 208.

Lorentz forces acting on the gradient coils system might be able to excite the lowest order vibrational mode. If the Lorentz forces act for example on the system 200 in the direction along the y-axis, whereby the direction of the Lorentz forces changes alternately from +y to −y and vice versa at a frequency that corresponds approximately to the frequency of the vibrational mode and whereby the phase relation between the Lorentz forces and the vibrational mode is appropriate, then the Lorentz forces are able to excite the vibrational mode. The gradient coils system might then perform large vibrations and produce a loud noise.

In contrast, if the phase relation between the Lorentz forces and the vibrational mode is inappropriate to excite the vibrational mode, no or nearly no noise will be generated as vibrations of the system 200 do not takes place. The case of Lorentz forces and the vibrational mode having a phase relation with respect to each other that is inappropriate to excite the vibrational mode is an example when the integral of the in-product of Lorentz forces and the vibrational mode along the gradient coils system corresponds to a value which is close to zero.

Another example is the case when the Lorentz forces are directed along the z-axis. Lorentz forces directed along the z-axis are not able to excite the mode even if they are large in magnitude. Then the above mentioned integral of the in-products of Lorentz forces and the vibrational mode taken over the gradient coils system corresponds to a value which is close to zero or even zero.

The two examples of how the above mentioned integral can become zero are used to demonstrate that if the integral becomes zero or is close to zero, then the Lorentz forces are not able to excite the vibrational mode even if the individual Lorentz forces acting on the gradient coils system are large in magnitude.

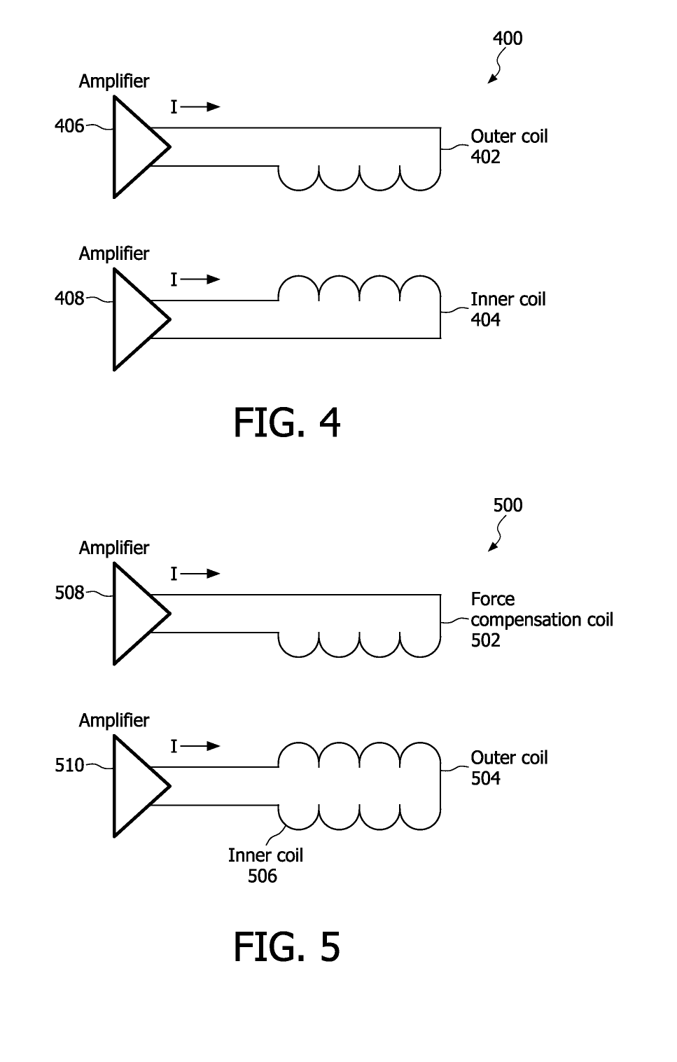

FIG. 4 depicts schematically an embodiment of a gradient coils system 400. A gradient coils system comprises an outer coil 402 and an inner coil 404. The outer coil 402 is electrically connected to a first amplifier 406 and the inner coil is electrically connected to a second amplifier 408. The inner coil 404 and the outer coil 402 are furthermore mechanically connected to each other by for example an epoxy resin which is not shown in FIG. 4. The first amplifier 406 provides a first electrical current to the outer coil. The second amplifier 408 provides a second current to the inner coil 404. As described before, the gradient coils system is used in a magnetic resonance imaging system to generate a magnetic gradient field. The magnetic gradient field that is to be generated in the examination volume of the magnetic resonance imaging system is usually provided by the inner coil 404. The outer coil 402 is usually employed to produce a magnetic field that cancels the magnetic field produced by the inner coil at the cryostat of the main magnet (the cryostat is not shown in FIG. 4). The second electrical current can then be provided to the inner coil 404 so that the desired magnetic gradient field is produced in the examination volume of the magnet. The first electrical current that is provided by the first amplifier 406 can then be set to an amplitude which does not lead to an optimal cancellation of the stray magnetic fields produced by the inner coil 404 at the cryostat, but that compensates the Lorentz forces acting on the gradient coils system so that vibrations of the gradient coils system are not excited.

FIG. 5 shows schematically another embodiment of a gradient coils system 500. The gradient coils system 500 comprises a force compensation coil 502, an outer coil 504 and an inner coil 506. The force compensation coil 502 is electrically connected to a first amplifier 508. The inner coil 506 and the outer coil 504 are electrically connected to each other. The inner and outer coil 504 and 506 are furthermore electrically connected to a second amplifier 510. The first amplifier 508 provides a first electrical current to the first compensation coil 502. The second amplifier 510 provides a second electrical current through the inner coil and the outer coil. The required first electrical current is small with respect to the second electrical current. Hence, a relative inexpensive low power amplifier can be employed as first amplifier 508.

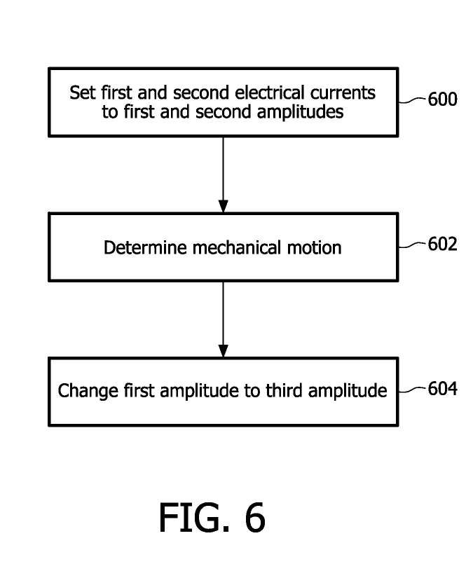

FIG. 6 shows a flow diagram that illustrates basic steps performed by a method of reducing the acoustic noise generated by a magnetic resonance imaging system, wherein the magnetic resonance imaging system comprises means for generating a static magnetic field in a examination volume of the magnetic resonance imaging system, a gradient coils system for generating a time varying magnetic gradient field in the examination volume, wherein the time varying magnetic gradient field is activated by a first electrical current and a second electrical current flowing through the gradient coils system. In step 600 of the method in accordance with the invention, the first electrical current is set to a first amplitude and the second electrical current is set to a second amplitude, wherein the first and second amplitudes are set in order to generate a desired time varying magnetic gradient field in the examination volume. In step 602, the mechanical motion of the gradient coils system is determined. In step 604, the first amplitude of the first electrical current is changed to a third amplitude so that the mechanical motion of the gradient coils system is minimized, wherein the mechanical motion is induced by the interaction of the electrical currents with the superposition of the magnetic gradient field and the static magnetic field.

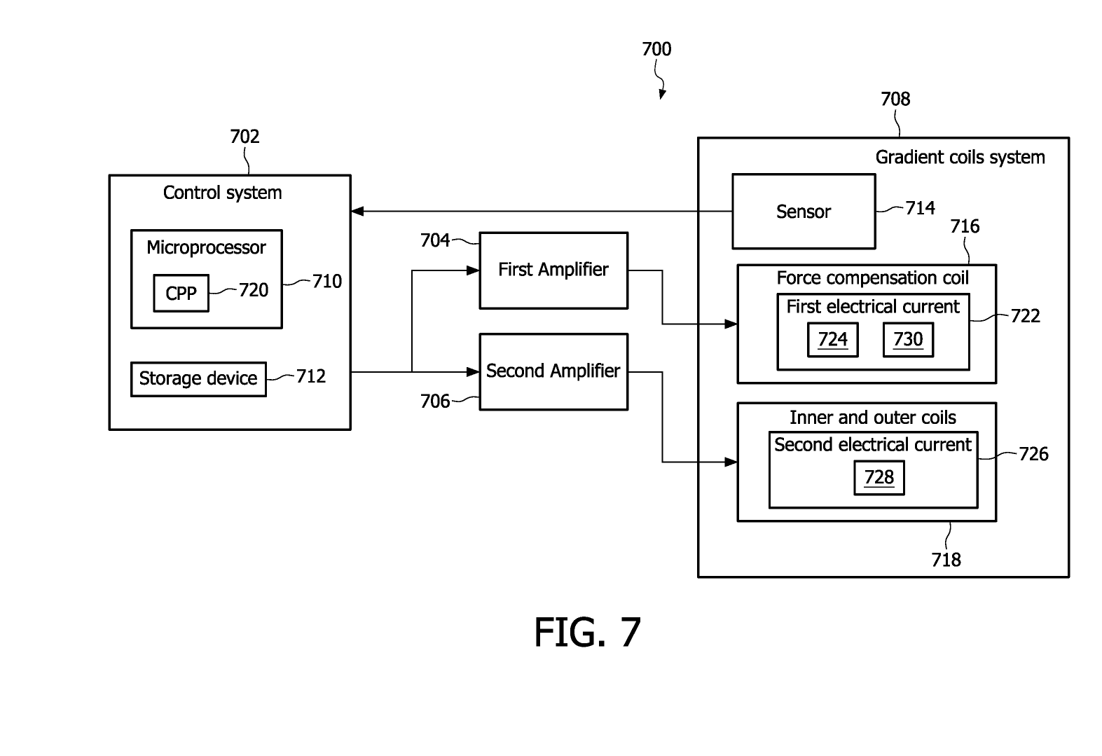

FIG. 7 shows a block diagram of a magnetic resonance imaging system 700. The magnetic resonance imaging system comprises a control system 702, a first amplifier 704, a second amplifier 706, and a gradient coils system 708. The control system 702 comprises a microprocessor 710 and a storage device 712. The gradient coils system 708 comprises a sensor 714, a force compensation coil 716, and an inner and outer coil 718. The inner and outer coils 718 and the force compensation coil 716 are mechanically connected with each other as they are for example embedded in an epoxy resin as described before. The sensor 714 is mounted to the gradient coils system 708. The sensor 714 can be an accelerometer and can therefore be used to determine the acceleration of the gradient coils system 708 when bouncing back and forth due to the vibration.

The control system 702 is connected to the first amplifier and to the second amplifier 704 and 706. The first amplifier 704 is furthermore electrically connected to the force compensation coil 716 and the second amplifier 706 is furthermore electrically connected to the inner and outer coils 718. The control system 702 is able to control the first and second amplifiers 704 and 706. The microprocessor 710 executes a computer program product 720 which is permanently stored on the storage device 712 and loaded into the microprocessor 710 for example at the start-up of the control system 702. The computer program product 720 comprises computer executable instructions by which the first amplifier 704 is controlled so that it provides a first electrical current 722 with a first amplitude 724 to the force compensation coil 716. Furthermore, the second amplifier 706 is controlled by the computer program product 720 so that it provides a second electrical current 726 with a second amplitude 728 to the inner and outer coils 718. The time-varying amplitudes of the first and the second electrical currents are chosen so that the desired time-varying magnetic gradient field is produced in the examination volume of the magnet and so that the generation of eddy currents in conducting surfaces of the MRI system, e.g. in the cryostat of the magnetic resonance imaging system, is minimized. The computer program product 720 is further able to read out the sensor 714. Thus it is able to determine a measure of the mechanical motion of the gradient coils system 708. The time-varying first amplitude of the first electrical current is then changed to a third time-varying amplitude 730 so that the mechanical motion of the gradient coils system is minimized.

MRI systems such as MRI system 700 are usually employed for a variety of examinations carried out on a patient. In each examination, predetermined time-varying magnetic gradient fields are required in the examination volume in order to be able to generate for example 3D images of the patient. The examinations are referred to as scans. As mentioned above, time-varying magnetic gradient fields that are required for a scan are known. Thus for each scan the first, second and third amplitudes 724, 728, and 730 (which might also be functions of time) of the first and second currents 722 and 726 can therefore be determined. The settings of the currents can be stored for example on the storage device 712 and the amplitudes 730 and 728 can then be applied during the corresponding scan. The first amplitude 724 can also be used in the case when a scan requires the optimal possible magnetic gradient field in the examination volume (when condition a) as mentioned above must be fulfilled). If the settings of the first and second electrical currents 722 and 726 are determined in advance and stored on the storage device, then the sensor 714 is not needed any more. Thus, feed-forward control can be employed in order to reduce the acoustic noise generated by the MRI system.

In another embodiment, the sensor can be a microphone that is mounted for example to the examination volume of the MRI system. The control system adjusts then the amplitude of the first electrical current so that the acoustic power detected by the microphone is at a minimum level.

In a further embodiment, a temperature sensor can be mounted to the gradient coils system. The temperature sensor is used to measure the temperature of the gradient coils system. A change of temperature of the gradient coils system causes a change of the mode shapes and of the oscillation frequency of the vibrational modes. The control system 702 changes then after detecting a change of temperature the first amplitude of the electrical current to the third amplitude so that the acoustic noise generated by the MRI system 700 is minimized.

The first electrical current 722 can generally be described as a superposition of a plurality of electrical currents. These currents are further referred to as sub-currents. Each sub-current of the plurality of currents has a specific frequency and specific (time-varying) amplitude.

As mentioned above, the lowest order bending mode is the dominant vibrational mode of the gradient coils system and the major source of noise. The lowest order bending mode has a specific oscillation frequency. Only sub-currents with frequencies that are close to the specific oscillation frequency of the lowest order bending mode are able to excite the lowest order bending mode efficiently. The resonance of the lowest order bending mode around the specific oscillation frequency has a certain line width. The line width can for example be measured by the so called full width half maximum (FWHM). A range can be set that defines if a frequency f_{sc} of a sub-current is close to the specific oscillation frequency f₀ by use of, e.g., the above mentioned FWHM. For example, the range can be given by twice the FWHM. Then the frequency f_{sc} is close to f₀ if the criterion

|f₀−f_{sc}|≦2·FWHM

is fullfilled.

In an embodiment of the invention, only the instantaneous amplitudes of the sub-currents with specific frequencies close to the oscillation frequency of the lowest order bending mode, e.g. that lie in the range defined above, are adapted by the control system 702 in order to minimize the noise. The amplitudes of the other sub-currents constituting the first current are adapted so that the desired time-varying magnetic gradient field is generated. Thus, in order to minimize the negative effect on imaging performance caused by the eddy currents induced at the cryostat, only the amplitudes of the sub-currents which are able to excite the lowest order bending mode efficiently are adjusted so that the noise generated by the gradient coils system is minimized. The time-varying amplitudes of the remaining sub-currents are adapted so that the desired magnetic field is generated at the examination volume.

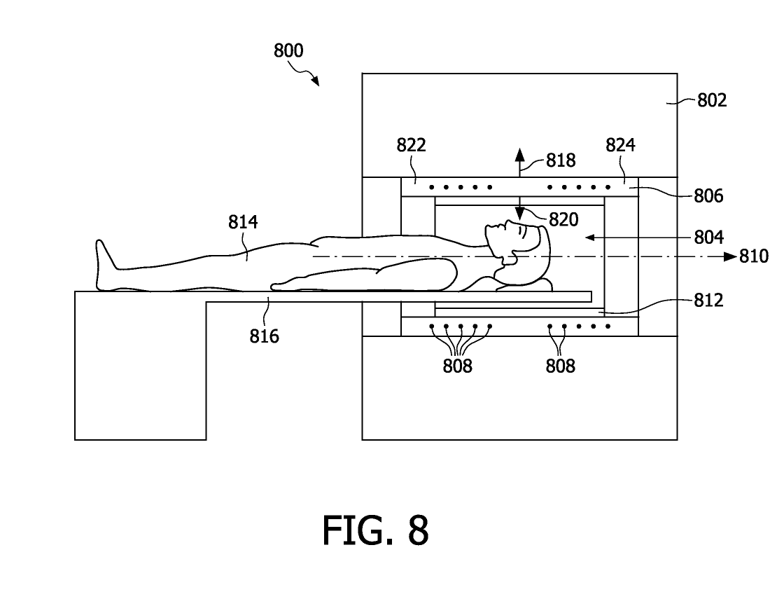

FIG. 8 shows a cross section through of a magnetic resonance system 800. The MRI system 800 comprises a main magnet 802 for generating a static magnetic field in the examination volume 804 of the MRI system. The MRI system 800 further comprises a gradient coils system 806 for generating time-varying gradient magnetic fields in the examination volume 804. The gradient coils system 806 comprises a plurality of windings, and as an example, cross sections of conductors 808 are indicated as black dots. Further, the gradient coils system 806 and the main magnet 802 are cylindrically symmetric with respect to a center axis 810. The MRI system 802 further comprises a high-frequency system 812 that emits radio-frequency signals into an examination subject, for example a patient 814, for triggering magnetic resonance signals and that picks up the generated magnetic resonance signals. The MRI system 800 has also a bearing device 816 on which the patient 814 is situated and by which the patient can be pushed in or pulled out of the examination space 804.

The gradient coils system 806 is able to perform mechanical vibrations, mainly in the dominant mode which corresponds to the first order bending mode. The arrows 818 and 820 illustrate the direction of the mechanical vibrations at locations, where the lowest order bending mode has an anti-node, whereas at locations 822 and 824, the lowest order mode has a node. The mechanical vibration is the major source of noise generated by the gradient coil system 806. The MRI system 800 in accordance with the invention is particularly advantageous as the acoustic output of the gradient coils system 806 is largely reduced due to an adaptation of the currents flowing through the gradient coils system and/or the design of the gradient coils system. The comfort of the patient 814 lying in the examination space is largely enhanced as he is exposed to less noise during his examination.

In the subsequent claims, reference signs have been incorporated in order to facilitate an understanding of the claims. Any reference in the claims shall however not be construed as limiting the scope.

### LIST OF REFERENCE NUMERALS

100 magnetic resonance imaging system
102 main magnet
104 gradient coils system
106 examination volume
108 center axis
110 coordinate system
112 first current
114 second current
116 magnetic gradient field
118 static magnetic field
120 static magnetic field
122 magnetic gradient field
124 resulting magnetic field
126 electrical current
128 Lorentz force
200 gradient coils system
202 coordinate system
204 outer coil
206 inner coil
208 epoxy resin
210 center axis
400 gradient coils system
402 outer coil
404 inner coil
406 first amplifier
408 second amplifier
500 gradient coils system
502 force compensation coil
504 outer coil
506 inner coil
508 first amplifier
510 second amplifier
700 magnetic resonance imaging system
702 control system
704 first amplifier
706 second amplifier
708 gradient coils system
710 microprocessor
712 storage device
714 sensor
716 force compensation coil
718 inner and outer coils
720 computer program product
722 first electrical current
724 first amplitude
726 second electrical current
728 second amplitude
730 third amplitude
800 MRI system
802 main magnet
804 examination volume
806 gradient coils system
808 conductors
810 center axis
812 high-frequency system
814 patient
816 bearing device
818 arrow indicting direction of oscillation at location of anti-node
820 arrow indicting direction of oscillation at location of anti-node
822 location
824 location

1. A magnetic resonance imaging system comprising:
means for generating a static magnetic field, and
a gradient coils system for generating a time-varying magnetic gradient field by use of a first electrical current and a second electrical current, said gradient coils system being located in said static magnetic field, said gradient coils system having a plurality of vibrational modes,
wherein Lorentz forces are generated along the gradient coils system due to the interaction of said first and/or said second electrical currents with the superposition of said static magnetic field and said magnetic gradient field, wherein said gradient coils system and/or said first electrical current are adapted so that the integral of the in-products of said Lorentz forces and of a vibrational mode of said plurality of vibrational modes is at a value close to zero, wherein said in-products are determined for all points of the gradient coils system, and wherein the integral is determined by summing the in-products determined for all points.

2. The magnetic resonance imaging system according to claim 1, wherein said gradient coils system comprises an inner coil and an outer coil, wherein said inner and outer coils are mechanically coupled, wherein said outer coil is operated by use of said first electrical current, wherein said inner coil is operated by use of said second electrical current, wherein said first electrical current is adapted so that the integral of the in-products of said Lorentz forces and the vibrational mode is at a value close to zero, wherein said in-products are determined for all points of the gradient coils system, and wherein the integral is determined by summing the in-products determined for all points.

3. The magnetic resonance imaging system of claim 2, further comprising
a first amplifier and a second amplifier, said first amplifier being electrically connected with said outer coil, said first amplifier providing said first electrical current, said second amplifier being electrically connected to said inner coil, said second amplifier providing said second electrical current.

4. The magnetic resonance imaging system according to claim 2, wherein said outer coil comprises a plurality of windings, and wherein said first amplifier is a low power amplifier.

5. The magnetic resonance imaging system according to claim 1, wherein said gradient coils system comprises an inner coil, an outer coil, and a force compensation coil, wherein the inner, outer and force compensation coil are mechanically connected, wherein said force compensation coil is operated by use of said first electrical current, wherein said inner and outer coils are operated by use of said second electrical current.

6. The magnetic resonance imaging system according to claim 5, wherein the force compensation coil is located in the vicinity of the outer coil on the opposite side of the inner coil.

7. The magnetic resonance imaging system according to claim 5, wherein the magnetic resonance imaging system further comprises a first amplifier and a second amplifier, wherein said inner and said outer coil are electrically connected with said second amplifier, wherein said force compensation coil is electrically connected with said first amplifier, wherein said first amplifier provides said first electrical current, and wherein said second amplifier provides said second electrical current.

8. The magnetic resonance imaging system according to claim 1, wherein said vibrational mode corresponds to the first order bending mode of said gradient coils system.

9. The magnetic resonance imaging system according to claim 1, wherein said gradient coils system comprises a an inner z-coil, an outer z-coil and a force compensation coil, wherein the inner z-coil, the outer z-coil and the force compensation coil are mechanically coupled, wherein said force compensation coil is operated by use of said first electrical current, wherein said inner and outer z-coils are operated by use of said second electrical current.

10. The magnetic resonance imaging system according to claim 9, wherein the magnetic resonance imaging system further comprises a first amplifier and a second amplifier, wherein said inner and outer z-coils are electrically connected with said second amplifier, wherein said force compensation coil is electrically connected with said first amplifier, wherein said first amplifier provides said first electrical current, and wherein said second amplifier provides said second electrical current.

11. The magnetic resonance imaging system according to claim 9, wherein said vibrational mode corresponds to the breathing mode of the gradient coils system, wherein the breathing mode is the dominant vibrational mode when magnetic gradient fields are generated by use of said z-coil.

12. The magnetic resonance imaging system according to claim 1, wherein said first and said second electrical currents are time-varying electrical currents.

13. The magnetic resonance imaging system according to claim 1, wherein the spatial distribution of the conductors of said gradient coils system is adapted.

14. The magnetic resonance imaging system according to claim 1, wherein the first electrical current is adaptable so that the in-product of said Lorentz forces distributed along the gradient coils system and of each vibrational mode is in essence zero on each geometrical point along the gradient coils system.

15. The magnetic resonance imaging system of claim 1, further comprising
a control system,
wherein the control system is able to adapt the first time-varying electrical current so that the integral of the in-products of said Lorentz forces and the vibrational mode is at a value close to zero, wherein said in-products are determined for all points of the gradient coils system, and wherein the integral is determined by summing the in-products determined for all points.

16. A gradient coils system for a magnetic resonance imaging system, wherein said magnetic resonance imaging system comprising means for generating a static magnetic field, and wherein said magnetic resonance imaging system is adapted to receive said gradient coils system so that the received gradient coils system is located in said static magnetic field, wherein said gradient coils system has a plurality of vibrational modes, wherein said gradient coils system is adapted to generate a magnetic gradient field in the magnetic resonance imaging system, wherein said magnetic gradient field is activated by a first electrical current and a second electrical current, wherein Lorentz forces are generated along the gradient coils system due to the interaction of said first and second electrical currents with the superposition of said static magnetic field and said magnetic gradient field, wherein said gradient coils system and/or said first electrical current are adapted so that so that the integral of the in-products of said Lorentz forces and a vibrational mode of said plurality of vibrational modes is at a value close to zero, wherein said in-products are determined for all points of the gradient coils system, and wherein the integral is determined by summing the in-products determined for all points.

17. A method of reducing acoustic noise generated by a magnetic resonance imaging system, said magnetic resonance imaging system comprising means for generating a static magnetic field in an examination volume of said magnetic resonance imaging system, and a gradient coils system for generating a time-varying magnetic gradient field in said examination volume, wherein said time-varying magnetic gradient field is activated by a first electrical current and a second electrical current flowing through the gradient coils system, said method comprising:
setting the first electrical current to a time-varying first amplitude and the second electrical current to a time-varying second amplitude, wherein the first and second amplitudes vary in time so that a desired time-varying magnetic gradient field is generated in said examination volume;
changing the first amplitude of said first electrical current to a time-varying third amplitude so that a mechanical motion of said gradient coils system is minimized, wherein said mechanical motion is induced by the interaction of said first and second electrical currents with the superposition of said magnetic gradient field and said static magnetic field.

18. The method of claim 17, wherein said gradient coils system comprises a dominant vibrational mode with a specific oscillation frequency, wherein the first current is a superposition of a plurality of currents, wherein each current of said plurality of currents has a specific frequency and a specific time-varying amplitude, wherein the specific time-varying amplitude of each current having a specific frequency which is close to the specific oscillation frequency is adapted so that the mechanical motion of said gradient coils system is minimized, and wherein the time-varying amplitudes of the remaining currents of said plurality of currents are adapted so that the desired time-varying magnetic gradient field is generated in said examination volume.

19. A computer program product for reducing acoustic noise generated by a magnetic resonance imaging system, said system comprising means for generating a static magnetic field in a examination volume of said magnetic resonance imaging system, and a gradient coils system for generating a time-varying magnetic gradient field in said examination volume, wherein said time-varying magnetic gradient field is activated by a first electrical current and a second electrical current flowing through the gradient coils system, said computer program product comprising computer executable instructions, said instructions being adapted to performing the steps:
setting the first electrical current to a first amplitude and the second electrical current to a second amplitude, wherein the first and second amplitudes are set in order to generate a desired time-varying magnetic gradient field in said examination volume;
changing the first amplitude of said first electrical current to a third amplitude so that a mechanical motion of said gradient coils system is minimized, wherein said mechanical motion is induced by the interaction of said first and second electrical currents with the superposition of said magnetic gradient field and said static magnetic field.
